(19) United States
(12) Patent Application Publication
(10) Pub. No.: US 2024/0398356 A1
(43) Pub. Date: Dec. 5, 2024

(54) NOTIFICATIONS BASED ON PATIENT AND CAREGIVER PROXIMITY

(71) Applicant: Hill-Rom Services, Inc., Batesville, IN (US)

(72) Inventors: Avner Halperin, Ramat Gan (IL); Zvika Shinar, Binyamina (IL)

(21) Appl. No.: 18/798,966

(22) Filed: Aug. 9, 2024

Related U.S. Application Data
(63) Continuation of application No. 16/129,878, filed on Sep. 13, 2018, now abandoned.
(60) Provisional application No. 62/559,568, filed on Sep. 17, 2017.

(51) Int. Cl. A61B 5/00; A61B 10/00; G16H 40/63; G16H 40/67; G16H 50/20

(52) U.S. Cl. CPC A61B 5/7275; A61B 5/4824; A61B 10/0012; G16H 40/63; G16H 40/67; G16H 50/20; A61B 2562/0219

(57) ABSTRACT

Apparatus is provided for monitoring a female subject. During a first menstrual cycle of the subject a computer processor predicts an upcoming ovulation of the subject, generates an output, on at least one user interface device, in response to predicting the upcoming ovulation, and receives from the subject, via the user interface device, an indication of one or more sensations that are sensed by the subject during a time period that is in temporal proximity to the predicted upcoming ovulation. During a second menstrual cycle of the subject, subsequent to the first menstrual cycle, the computer processor predicts an upcoming ovulation of the subject, at least partially based upon the indication received from the subject during the first menstrual cycle, and generates, on the user interface device, an output in response to predicting the upcoming ovulation of the subject. Other applications are also described.

### CROSS-REFERENCE TO RELATED APPLICATIONS

The present application is a continuation of U.S. application Ser. No. 16/129,878, filed Sep. 13, 2018, now abandoned, which claims the benefit of U.S. Provisional Application 62/559,568, filed Sep. 17, 2017, each of which are hereby incorporated by reference herein in their entirety.

### FIELD OF EMBODIMENTS OF THE INVENTION

The present invention relates generally to monitoring a subject. Specifically, some applications of the present invention relate to monitoring a female subject, and/or an infirm subject.

### BACKGROUND

There is great variation in the lengths of women's menstrual cycles. It is often the case that women would like to know the current phase of their menstrual cycle. Of particular interest to many is knowledge of when they are in the “fertile window” which occurs from approximately five days before ovulation until two days after ovulation. Typically, urine tests, calendar-based methods, and symptoms-based methods (in which parameters such as cervical mucus, cervical position, and basal body temperature are measured) are used for such determinations.

### SUMMARY OF EMBODIMENTS

In accordance with some applications of the present invention, during a first menstrual cycle of a female subject, a sensor monitors the subject, and generates a sensor signal in response to the monitoring. A computer processor receives the sensor signal, and analyzes the sensor signal. At least partially in response to the analyzing, the computer processor predicts an upcoming ovulation of the subject. Typically, the computer processor generates an output, in response to predicting the upcoming ovulation of the subject. For some applications, the computer processor predicts an upcoming ovulation of the subject, even in the absence of the computer processor receiving an input from the sensor signal. For example, the computer processor may receive an input from the subject that is indicative of dates and times at which menstrual events occurred (e.g., when her menses occurred), and/or lengths of her menstrual cycles.

Subsequently, the computer processor receives indications of one or more sensations that are sensed by the subject during a given time period that is in temporal vicinity to the predicted upcoming ovulation (e.g., from between 6 and 12 hours and/or between 1 and 3 days prior to the predicted upcoming ovulation, until 6 and 12 hours and/or between 1 and 3 days after the predicted ovulation). For example, such sensations may include cramping and/or pain (e.g., cramping and/or pain on one side of the pelvis). For some applications, such sensations include ovulation pain, which is sometimes referred to as mittelschmerz. For some applications, other physical sensations such as changes in cervical fluid, spotting, changes in fluid production during sexual intercourse, breast tenderness, abdominal bloating, increased sex drive, nausea, headaches (e.g., migraines), and/or a heightened sense of smell, taste and/or vision may also be entered by the user.

For some applications, the computer processor prompts the subject to input such indications. For example, the computer processor may prompt the subject to answer questions regarding sensations that are sensed by the subject during the given time period in temporal vicinity to the predicted upcoming ovulation. For some applications, such prompts may include suggestions that the subject should focus on sensing a given sensation. Alternatively or additionally, the subject may input indications of sensations that she senses in the given period, in the absence of prompting by the computer processor.

It is noted that, in general, some women have the ability to physically feel changes that their bodies undergo as a result of ovulation. By the user inputting indications of sensations that she feels around the time of ovulation, the computer processor is able to use such sensations as additional data for predicting future upcoming ovulation events, as described in further detail hereinbelow. Moreover, as described hereinabove, for some applications, the computer processor prompts the user to input information regarding sensations that she is feeling. In this manner, the computer processor trains the user to be sensitive to changes in her body around the time of ovulation, such that her ability to sense, on her own, that she is undergoing ovulation, is enhanced.

Typically, in a subsequent menstrual cycle, the computer processor receives the sensor signal, and/or receives an input from the subject that is indicative of the timings of menstrual events, as described hereinabove. The computer typically predicts an upcoming ovulation by analyzing (a) the sensor signal received during the second menstrual cycle, and/or the input from the subject received during the second menstrual cycle, and (b) the feedback that the computer processor received during the first menstrual cycle, by means of the indications received from the subject during the first menstrual cycle. Typically, the computer processor generates an output in response to predicting the upcoming ovulation of the subject. For some applications, the computer processor generates an output indicating the time period of the upcoming ovulation. Typically, subsequent to generating the output indicating the time period of the upcoming ovulation, the computer processor again receives, from the subject, indications of one or more sensations that are sensed by the subject that are indicative of the subject undergoing ovulation. As described hereinabove, the subject may be prompted to answer questions regarding sensations that are sensed by the subject, and/or the subject may input such indications to the computer processor without being prompted to do so by the computer processor.

The above-described process of the computer predicting the subject's ovulation, and, in turn, receiving inputs from the subject that are indicative of the subject undergoing ovulation, are iteratively repeated over subsequent menstrual cycles. In this manner, typically, the computer processor trains the user to be more sensitive to her ovulation sensations and, in turn, the user trains the computer processor to become more accurate in predicting the ovulation. Typically, when a woman feels pains associated with ovulation, this means that she is toward the end of her fertile period. For some applications, training the computer processor to become more accurate in predicting her ovulation enables the user to utilize more of her fertile days than if she were just to sense ovulation herself. On the other hand, by learning to sense ovulation herself, the user becomes better equipped to train the computer processor.

For some applications, apparatus and methods are provided for use with an infirm subject who has a telecommunications device, such as a phone. A computer processor detects whether any of a set of telecommunication devices associated with the people other than the subject is disposed within a given distance of the subject's telecommunication device. In response to detecting that, over a given time period, none of the set of telecommunication devices associated with the people other than the subject is disposed within the given distance of the first telecommunication device, the computer processor generates an alert.

Typically, the distance is set such that if the subject's telecommunications device is in the subject's home or room, the computer processor detects whether any of the set of telecommunication devices associated with the people other than the subject is disposed within the home or the room. For example, in this manner, if the subject lives in a private home, the computer processor is able to detect whether the subject's home has been visited by any of the people with whom the further set of telecommunication devices are associated. Or, if the subject lives in a care-home or is in a hospital ward, the computer processor is able to detect whether the subject's room or ward has been visited by any of the people with whom the further set of telecommunication devices are associated. For some applications, the computer processor detects whether any of the further devices are within the given distance using a communications protocol such as Bluetooth, Zigbee, and/or a similar protocol.

For some applications, the computer processor detects whether any telecommunications devices belonging to any person other than the subject are disposed within the given distance of the subject's telecommunications device. If no telecommunications devices belonging to any person other than the subject are disposed within the given distance of the subject's telecommunications device over a given time period, an alert is generated, since this indicates that the infirm subject has been left alone over the given time period.

Alternatively or additionally, a set of telecommunication devices that are associated with a given set of people other than the subject is designated. For example, such people may include friends, relatives, and/or caregivers of the infirm subject. Typically, telecommunications devices belonging to a set of people, at least one of whom is scheduled to visit the subject once every given time period (e.g., once a day, once a week, or once every few hours), is designated. The computer processor is provided with identifying information regarding devices that are associated with the set of people, as well as an indication of the desired time period.

For some applications, the computer processor receives designations of respective telecommunication devices belonging to the set of telecommunication devices, as being associated with respective people. Typically, the computer processor receives an indication that one of the set of telecommunication devices associated with the people other than the subject is disposed within a given distance of the subject's telecommunication device. For example, the computer processor may be configured to detect that one of the telecommunications devices is at a distance that is indicative of the other person being at the door to the subject's room, or at the front door of the subject's home. For some applications, the telecommunications devices communicate with the subject's telecommunications devices via a communications protocol such as Bluetooth, Zigbee, etc. Typically, at least partially in response to receiving the indication, the computer processor generates an output on the subject's telecommunication device, indicating the identity of the person associated with the telecommunication device that is within the given distance of the first telecommunication device.

For example, in response to someone ringing at the subject's front door, the subject may check an application on his/her phone to check whether the person at her door is one of the people who is designated as being one of his/her caregivers, e.g., a child, a grandchild, a nurse, etc. A computer processor associated with the application on his/her phone will check whether there is a telecommunications device within a given distance of the subject's phone that belongs to one of a designated group of caregivers. In response to detecting that there is such a device within the given distance, the computer processor drives the application to display a picture of the caregiver to whom the device belongs, and/or to display text on the subject's phone (or to generate an audio output) indicating the identity of the person to whom the telecommunications device belongs.

There is therefore provided, in accordance with some applications of the present invention, apparatus for monitoring a female subject, the apparatus comprising:
at least one user interface device; and
a computer processor, configured:
during a first menstrual cycle of the subject:
to predict an upcoming ovulation of the subject,
to generate an output, on the at least one user interface device, in response to predicting the upcoming ovulation of the subject, and
to receive from the subject, via the at least one user interface device, an indication of one or more sensations that are sensed by the subject during a given time period that is in temporal proximity to the predicted upcoming ovulation, and
during a second menstrual cycle of the subject that is subsequent to the first menstrual cycle:
to predict an upcoming ovulation of the subject, at least partially based upon the indication received from the subject during the first menstrual cycle, and
to generate an output, on the at least one user interface device, in response to predicting the upcoming ovulation of the subject.

In some applications,
the apparatus further includes a sensor, configured to monitor the subject, and to generate a sensor signal in response to the monitoring, and
the computer processor is configured:
during the first menstrual cycle:
to receive the sensor signal, and
to predict an upcoming ovulation of the subject in response to analyzing the sensor signal, and
during the second menstrual cycle:
to receive the sensor signal, and
to predict the upcoming ovulation of the subject, by analyzing the sensor signal in combination with the indication received from the subject during the first menstrual cycle.

In some applications,
the computer processor is configured:
during the first menstrual cycle:
to receive an input from the subject that is indicative of timings of one or more menstrual events of the subject, and
to predict an upcoming ovulation of the subject in response to the input, and
during the second menstrual cycle:
to receive an input from the subject that is indicative of timings of one or more menstrual events of the subject, and
to predict the upcoming ovulation of the subject, by analyzing the input in combination with the indication received from the subject during the first menstrual cycle.

In some applications, the computer processor is configured:
during the second menstrual cycle, to receive from the subject, via the at least one user interface device, an indication of one or more sensations that are sensed by the subject during a given time period that is in temporal proximity to the predicted upcoming ovulation, and
during further menstrual cycles of the subject that are subsequent to the second menstrual cycle, to predict upcoming ovulations of the subject, at least partially based upon the indications received from the subject, during the first and second menstrual cycles.

In some applications, the computer processor is configured to:
receive from the subject, via the at least one user interface device, indications of confidence levels associated with the sensations sensed by the subject during the first and second menstrual cycles, and
during the further menstrual cycles of the subject, to predict upcoming ovulations of the subject, at least partially based upon the indications received from the subject during the first and second menstrual cycles, in combination with the associated confidence level indications.

There is further provided, in accordance with some applications of the present invention, apparatus for use with a first telecommunication device that is associated with a subject requiring care, and a set of telecommunication devices that are associated with people other than the subject, the apparatus including:
at least one computer processor configured:
to detect whether any of the set of telecommunication devices associated with the people other than the subject is disposed within a given distance of the first telecommunication device; and
in response to detecting that, over a given time period, none of the set of telecommunication devices associated with the people other than the subject is disposed within the given distance of the first telecommunication device, to generate an alert.

In some applications, the set of telecommunication devices associated with the people other than the subject includes telecommunication devices associated with any person other than the subject, and the at least one computer processor is configured to generate the alert in response to detecting that, over a given time period, no telecommunication device associated with any person other than the subject is disposed within the given distance of the first telecommunication device.

In some applications, the set of telecommunication devices associated with the people other than the subject includes telecommunication devices associated with a set of caregivers that are assigned the subject, and the at least one computer processor is configured to generate the alert in response to detecting that, over a given time period, none of the set of telecommunication devices associated with the set of caregivers that are assigned to the subject is disposed within the given distance of the first telecommunication device.

There is further provided, in accordance with some applications of the present invention, apparatus for use with a first telecommunication device that is associated with a subject requiring care, and a set of telecommunication devices associated with people other than the subject, the apparatus including:
at least one computer processor configured:
to receive designations of respective telecommunication devices belonging to the set of telecommunication devices, as being associated with respective people;
to receive an indication that one of the telecommunication devices belonging to the set of telecommunication devices is within a given distance of the first telecommunication device; and
at least partially in response thereto, to generate an output on the first telecommunication device, indicating an identity of the person associated with the telecommunication device that is within a given distance of the first telecommunication device.

The present invention will be more fully understood from the following detailed description of applications thereof, taken together with the drawings, in which:

### BRIEF DESCRIPTION OF THE DRAWINGS

FIG. 1 is a schematic illustration of apparatus for monitoring a female subject, in accordance with some applications of the present invention;

FIG. 2 is a flowchart showing steps that are performed by a computer processor in order to detect that the subject is ovulating, in accordance with some applications of the present invention;

FIG. 3 is a schematic illustration of an infirm subject holding a phone, in accordance with some applications of the present invention;

FIG. 4 is a flowchart showing steps that are performed by a computer processor in order to aid the infirm subject, in accordance with some applications of the present invention;

FIG. 5 is a flowchart showing steps that are performed by a computer processor in order to aid the infirm subject, in accordance with some applications of the present invention; and

FIG. 6 is a schematic illustration of a medication delivery pump that is used with a subject monitoring sensor, in accordance with some applications of the present invention.

### DETAILED DESCRIPTION OF EMBODIMENTS

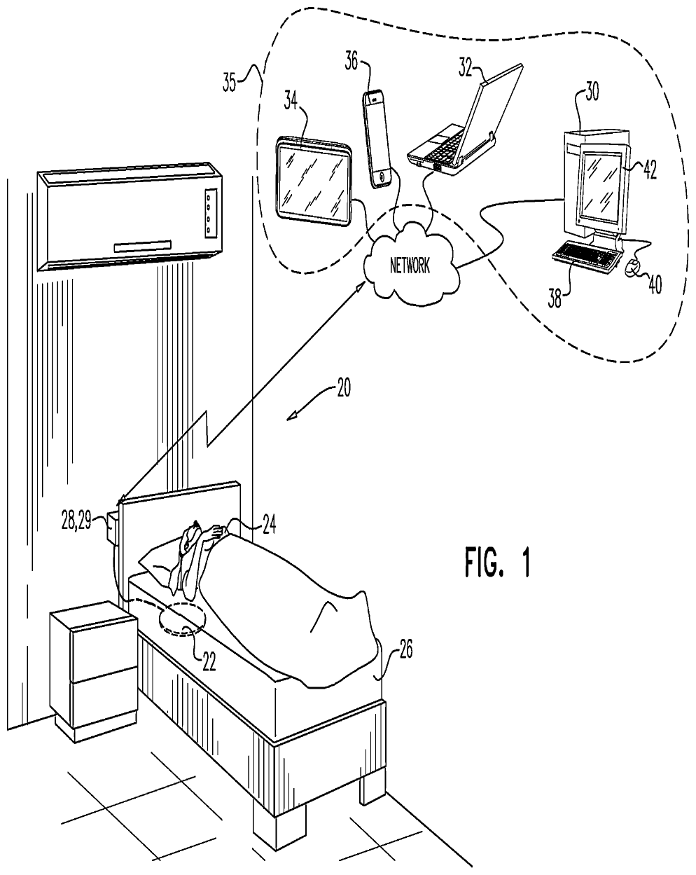

Reference is made to FIG. 1, which is a schematic illustration of subject-monitoring apparatus 20, in accordance with some applications of the present invention. Apparatus 20 is generally used to monitor a subject 24, while he or she is in his or her bed in a home setting. For some applications, the subject-monitoring apparatus is used in a hospital setting.

Subject-monitoring apparatus 20 comprises a sensor 22 (e.g., a motion sensor) that is configured to monitor subject 24. Sensor 22 may be a motion sensor that is similar to sensors described in U.S. Pat. No. 8,882,684 to Halperin, which is incorporated herein by reference. The term “motion sensor” refers to a sensor that senses the subject's motion (e.g., motion due to the subject's cardiac cycle, respiratory cycle, or large-body motion of the subject), while the term “sensor” refers more generally to any type of sensor, e.g., a sensor that includes an electromyographic sensor and/or an imaging sensor.

Typically, sensor 22 includes a sensor that performs monitoring of the subject without contacting the subject or clothes the subject is wearing, and/or without viewing the subject or clothes the subject is wearing. For example, the sensor may perform the monitoring without having a direct line of sight of the subject's body, or the clothes that the subject is wearing, and/or without any visual observation of the subject's body, or the clothes that the subject is wearing. Further typically, the sensor performs monitoring of the subject without requiring subject compliance (i.e., without the subject needing to perform an action to facilitate the monitoring that would not have otherwise been performed). It is noted that, prior to the monitoring, certain actions (such as purchasing the sensor, placing the sensor under the subject's mattress, downloading software for use with the subject-monitoring apparatus, and/or configuring software for use with the subject-monitoring apparatus) may need to be performed. The term “without requiring subject compliance” should not be interpreted as excluding such actions. Rather the term “without requiring subject compliance” should be interpreted as meaning that, once the sensor has been purchased, placed in a suitable position and activated, the sensor can be used to monitor the subject (e.g., to monitor the subject during repeated monitoring sessions), without the subject needing to perform any actions to facilitate the monitoring that would not have otherwise been performed.

For some applications, sensor 22 is disposed on or within the subject's bed, and configured to monitor the subject automatically, while the subject is in their bed. For example, sensor 22 may be disposed underneath the subject's mattress 26, such that the subject is monitored while she is lying upon the mattress, and while carrying out her normal sleeping routine, without the subject needing to perform an action to facilitate the monitoring that would not have otherwise been performed. For some applications, sensor 22 is a sensor that does contact the subject's and/or clothes that the subject is wearing, and for some applications, sensor 22 is placed in a position other than underneath the subject's mattress.

A computer processor 28 analyzes the signal from sensor 22. Typically, computer processor 28 communicates with a memory 29. For some applications, computer processor 28 is embodied in a desktop computer 30, a laptop computer 32, a tablet device 34, a smartphone 36, and/or a similar device that is programmed to perform the techniques described herein (e.g., by downloading a dedicated application or program to the device), such that the computer processor acts as a special-purpose computer processor. For some applications, as shown in FIG. 1, computer processor 28 is a dedicated computer processor that receives (and optionally analyzes) data from sensor 22, and communicates with computer processors of one or more of the aforementioned devices, which act as external devices.

For some applications, the subject (or another person, such as a care-giver) communicates with (e.g., sends data to and/or receives data from) computer processor 28 via a user interface device 35. As described, for some applications, computer processor is embodied in a desktop computer 30, a laptop computer 32, a tablet device 34, a smartphone 36, and/or a similar device that is programmed to perform the techniques described herein. For such applications, components of the device (e.g., the touchscreen, the mouse, the keyboard, the speakers, the screen) typically act as user interface device 35. Alternatively, as shown in FIG. 1, computer processor 28 is a dedicated computer processor that receives (and optionally analyzes) data from sensor 22. For some such applications, the dedicated computer processor communicates with computer processors of one or more of the aforementioned external devices (e.g., via a network), and the user interfaces of the external devices (e.g., the touchscreen, the mouse, the keyboard, the speakers, the screen) are used by the subject, as user interface device 35, to communicate with the dedicated computer processor and vice versa. For some applications, in order to communicate with computer processor 28, the external devices are programmed to communicate with the dedicated computer processor (e.g., by downloading a dedicated application or program to the external device).

For some applications, the user interface includes an input device such as a keyboard 38, a mouse 40, a joystick (not shown), a touchscreen device (such as smartphone 36 or tablet device 34), a touchpad (not shown), a trackball (not shown), a voice-command interface (not shown), and/or other types of user interfaces that are known in the art. For some applications, the user interface includes an output device such as a display (e.g., a monitor 42, a head-up display (not shown) and/or a head-mounted display (not shown)), and/or a different type of visual, text, graphics, tactile, audio, and/or video output device, e.g., speakers, headphones, smartphone 36, or tablet device 34. For some applications, the user interface acts as both an input device and an output device. For some applications, the processor generates an output on a computer-readable medium (e.g., a non-transitory computer-readable medium), such as a disk, or a portable USB drive.

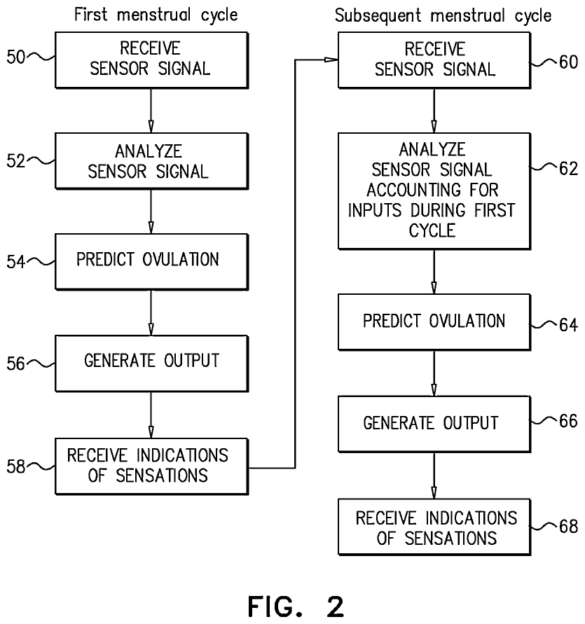

FIG. 2 is a flowchart showing steps that are performed by a computer processor in order to detect that the subject is ovulating, in accordance with some applications of the present invention.

For some applications, during a first menstrual cycle of the subject, sensor 22 monitors the subject, and generates a sensor signal in response to the monitoring. In a first step 50, computer processor 28 receives the sensor signal, and in a second step 52, the computer processor analyzes the sensor signal. In step 54, at least partially in response to the analyzing, the computer processor predicts an upcoming ovulation of the subject. Typically, the computer processor predicts the upcoming ovulation using techniques as described in US 2016/0058428 to Shinar and/or in US 2016/0058429 to Shinar, both of which applications are incorporated herein by reference. For example, the computer processor may identify an aspect of the sensor signal, such as a cardiac-related aspect of the sensor signal, and/or a respiration-related aspect of the sensor signal, and may perform the identification of the subject's state in response thereto.

For some applications, the subject's heart rate is identified. For example, an average heart rate over a period of time (e.g., an average heart rate over a sleeping session) may be identified, and, in response to ascertaining that the identified heart rate is greater than the baseline heart rate, the computer processor may identify that the subject is within a given amount of time (e.g., less than two days) of ovulation. In general, this output may help the subject with her fertility planning. In some applications, the computer processor uses the average heart rate of a previous sleeping session as a baseline, and in response to the identified average heart rate being greater than this baseline, the computer processor predicts the upcoming ovulation. In some applications, the computer processor predicts the upcoming ovulation, even in response to the identified heart rate being less than five heartbeats-per-minute greater than the baseline heart rate.

Alternatively or additionally to identifying a cardiac-related aspect of the sensor signal, the computer processor may identify a respiration-related aspect of the sensor signal, such as a respiratory rate of the subject. (For example, the computer processor may identify an average respiratory rate of the subject during a sleeping session of the subject.) In general, respiratory rate, like heart rate, typically rises to an elevated level at around the time of ovulation, and typically remains at the elevated level only if the subject becomes pregnant. Therefore, for example, the computer processor may identify the current phase of the menstrual cycle of the subject, by comparing the identified respiratory rate to a baseline respiratory rate. The use of the respiration-related aspect of the sensor signal may supplement, or alternatively, take the place of, the use of the cardiac-related aspect of the sensor signal.

In some applications, the identified aspect of the sensor signal includes a heart rate variability (HRV) signal, and the computer processor predicts an upcoming ovulation of the subject in response to the HRV signal, e.g., in response to an aspect of a component of the power spectrum of the HRV signal.

For some applications, the computer processor predicts an upcoming ovulation of the subject, even in the absence of the computer processor receiving an input from the sensor signal. That is to say, step 54 may be performed without steps 50 and 52 necessarily being performed. For example, the computer processor may receive an input from the subject that is indicative of dates and times at which menstrual events occurred (e.g., when her menses occurred), and/or lengths of her menstrual cycles.

In step 56, the computer processor generates an output (typically, via one or more user interface devices 35), in response to predicting the upcoming ovulation of the subject. In step 58, the computer processor receives indications of one or more sensations that are sensed by the subject during a given time period that is in temporal vicinity to the predicted upcoming ovulation (e.g., from between 6 and 12 hours and/or between 1 and 3 days prior to the predicted upcoming ovulation, until between 6 and 12 hours and/or between 1 and 3 days after the predicted ovulation). For example, such sensations may include cramping and/or pain (e.g., cramping and/or pain on one side of the pelvis). For some applications, such sensations include ovulation pain, which is sometimes referred to as mittelschmerz. For some applications, other physical sensations such as changes in cervical fluid, spotting, changes in fluid production during sexual intercourse, breast tenderness, abdominal bloating, increased sex drive, nausea, headaches (e.g., migraines), and/or a heightened sense of smell, taste and/or vision may also be entered by the user.

Typically, the subject inputs the indications into the computer processor via one or more user interface devices 35. For some applications, the computer processor prompts the subject to input such indications. For example, the computer processor may prompt the subject to answer questions regarding sensations that are sensed by the subject during the given time period that is temporal vicinity to the predicted upcoming ovulation. For some applications, such prompts may include suggestions that the subject should focus on sensing a given sensation. Alternatively or additionally, the subject may input indications of sensations that she senses in the given period in the absence of prompting by the computer processor.

It is noted that, in general, some women have the ability to physically feel changes that their bodies undergo as a result of ovulation. By the user inputting indications of sensations that she feels around the time of ovulation, the computer processor is able to use such sensations to as additional data for predicting future upcoming ovulation events, as described in further detail hereinbelow. Moreover, as described hereinabove, for some applications, the computer processor prompts the user to input information regarding sensations that she is feeling. In this manner, the computer processor trains the user to be sensitive to changes in her body around the time of ovulation, such that her ability to sense, on her own, that she is undergoing ovulation is enhanced.

Typically, in a subsequent menstrual cycle, in step 60, the computer processor receives the sensor signal. In step 62, the computer processor analyzes the sensor signal in combination with the feedback that the computer processor received from the subject regarding the ovulation prediction in the first menstrual cycle. For example, if the user was able to detect her ovulation pain in the first cycle, that detection may be used to fine tune and/or calibrate an ovulation prediction algorithm that is run by the computer processor. For example, if in the previous cycle the user sensed ovulation pain two days after a drop in average heart rate in deep sleep, then in the subsequent cycle, the computer processor may predict the ovulation to be two days after a similar drop in average heart rate. Additionally, the computer processor may be configured to receive an input that is indicative a confidence level of the user for her sensing of the actual ovulation, in respective menstrual cycles. Then, in subsequent cycles, the predicted delay in ovulation with respect to a change in heart rate may be calculated based upon a weighted average of the delay that was measured in previous cycles, with the confidence levels (or a function of the confidence level) associated with the user's inputs in respective previous cycles being used in the weighted average calculation.

As described hereinabove with reference to step 54, for some applications, step 62 is performed even in the absence of the computer processor receiving an input from the sensor signal. For example, the computer processor may receive an input from the subject that is indicative of dates and times at which menstrual events occurred (e.g., when her menses occurred), and/or lengths of her menstrual cycles, and the computer processor analyzes the input in combination with the feedback that the computer processor received from the subject regarding the ovulation prediction in the first menstrual cycle.

In step 64, the computer predicts an upcoming ovulation by analyzing (a) the sensor signal received during the second menstrual cycle and/or the input from the subject received during the second menstrual cycle, in combination with (b) the feedback that the computer processor received during the first menstrual cycle, by means of the indications received from the subject during the first menstrual cycle.

In step 66, the computer processor generates an output in response to predicting the upcoming ovulation of the subject. For some applications, the computer processor generates an output indicating the time period of the upcoming ovulation.

In step 68, subsequent to generating the output indicating the time period of the upcoming ovulation, the computer processor again receives, from the subject, indications of one or more sensations that are sensed by the subject that are indicative of the subject undergoing ovulation. As described hereinabove, the subject may be prompted to answer questions regarding sensations that are sensed by the subject, and/or the subject may input such indications to the computer processor without being prompted to do so by the computer processor.

The above-described process of the computer predicting the subject's ovulation, and, in turn, receiving inputs from the subject that are indicative of the subject undergoing ovulation, is typically iteratively repeated over subsequent menstrual cycles. Typically, in each subsequent cycle, ovulation is predicted based upon (a) the user's inputs from previous cycles being used in combination with (b) the sensor signal received during the current cycle, and/or the user's input during the current cycle regarding timings of menstrual events. In this manner, typically, the computer processor trains the user to be more sensitive to her ovulation sensations and, in turn, the user trains the computer processor to become more accurate in predicting the ovulation. Typically, when a woman feels pains associated with ovulation, this means that she is toward the end of her fertile period. For some applications, training the computer processor to become more accurate in predicting her ovulation enables the user to utilize more of her fertile days that if she were just to sense the ovulation herself. On the other hand, by learning to sense ovulation herself, the user becomes better equipped to train the computer processor.

It is noted that although the computer processor has been described as predicting an ovulation events, for some applications, similar algorithms are performed by the computer processor for determining that the subject is currently undergoing ovulation, mutatis mutandis. For such applications, the computer processor determines which sensations that sensed by the subject are indicative of the subject currently undergoing ovulation, rather than being indicative of the subject's ovulation being upcoming within a given time period.

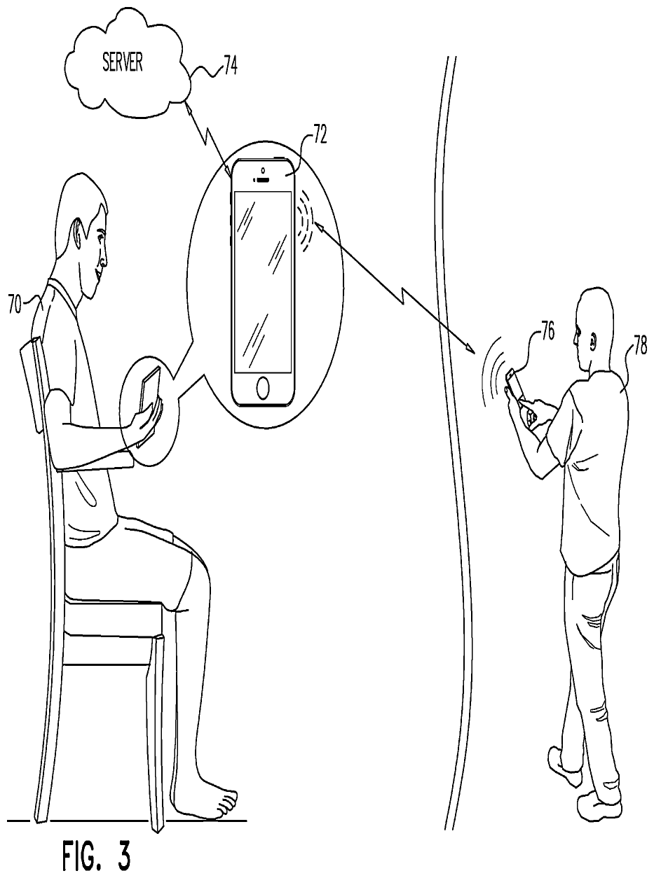

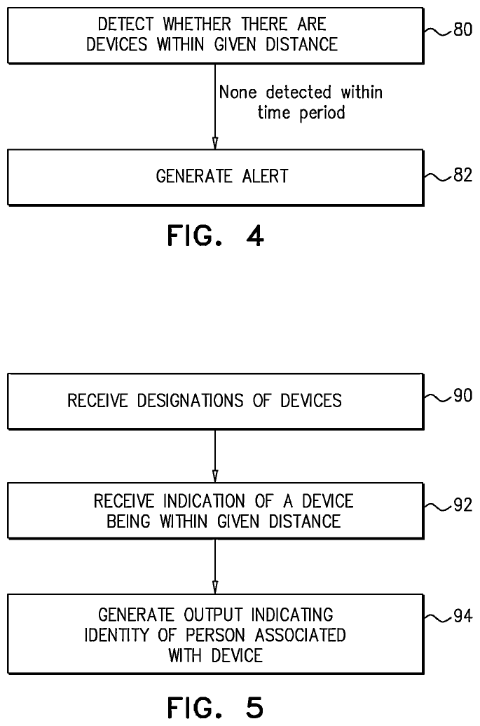

Reference is now made to FIG. 3, which is a schematic illustration of an infirm subject 70 holding a telecommunications device 72, such as a phone as shown, in accordance with some applications of the present invention. Alternatively or additionally, the telecommunications device is a computer, a laptop computer, a tablet device, etc. Typically, the telecommunications device includes a computer processor 28, which is generally as described hereinabove. Reference is also made to FIG. 4, which is a flowchart showing steps that are performed by the computer processor in order to aid the infirm subject, in accordance with some applications of the present invention.

Typically, in a first step 80, a computer processor (such as the computer processor of telecommunications device 72 of subject 70, or a computer processor associated with a remote server 74) detects whether any of the set of telecommunication devices associated with the people other than the subject is disposed within a given distance of the subject's telecommunication device. In a second step 82, in response to detecting that, over a given time period, none of the set of telecommunication devices associated with the people other than the subject is disposed within the given distance of the first telecommunication device, the computer processor generates an alert.

Typically, the distance is set such that if the subject's telecommunications device is in the subject's home or room, the computer processor detects whether any of the set of telecommunication devices associated with the people other than the subject is disposed within the home or the room. For example, in this manner, if the subject lives in a private home, the computer processor is able to detect whether the subject's home has been visited by any of the people with whom the set of telecommunication devices is associated. Or, if the subject lives in a care-home or is in a hospital ward, the computer processor is able to detect whether the subject's room or ward has been visited by any of the people with whom the set of telecommunication devices is associated. For some applications, the computer processor detects whether any of the further devices are within the given distance using a communications protocol such as Bluetooth, Zigbee, and/or a similar protocol.

For some applications, the computer processor detects whether any telecommunications devices belonging to any person other than the subject are disposed within the given distance of the subject's telecommunications device. If no telecommunications devices belonging to any person other than the subject are disposed within the given distance of the subject's telecommunications device over a given time period, an alert is generated, since this indicates that the infirm subject has been left alone over the given time period,

Alternatively or additionally, a set of telecommunication devices that are associated with a given set of people other than the subject is designated. For example, such people may include friends, relatives, and/or caregivers of the infirm subject. Typically, telecommunications devices belonging to a set of people, at least one of whom is scheduled to visit the subject once every given time period (e.g., once a day, once a week, or once every few hours), is designated. The computer processor is provided with identifying information regarding devices that are associated with the set of people, as well as an indication of the desired time period.

For some applications, the alert is generated on a telecommunications device (e.g., a phone) of the subject's primary caregiver. For example, the subject's child may be designated as the primary caregiver, and a nurse or the subject's grandchild may be scheduled to visit the subject once every afternoon. In response to detecting that the subject has not been visited by the nurse or the subject's grandchild one afternoon, an alert will be generated on the telecommunications device of the subject's child. Alternatively or additionally, the alert is generated at a monitoring center.

For some applications, the computer processor is configured to detect whether a telecommunications device other than a designated set of telecommunications devices is within a given distance of the subject's telecommunications device 72, and to generate an alert in response thereto. For example, in this manner, the computer processor may be configured to detect that there is someone present in the subject's home or room other than a predesignated set of caregivers, and to generate an alert in response thereto.

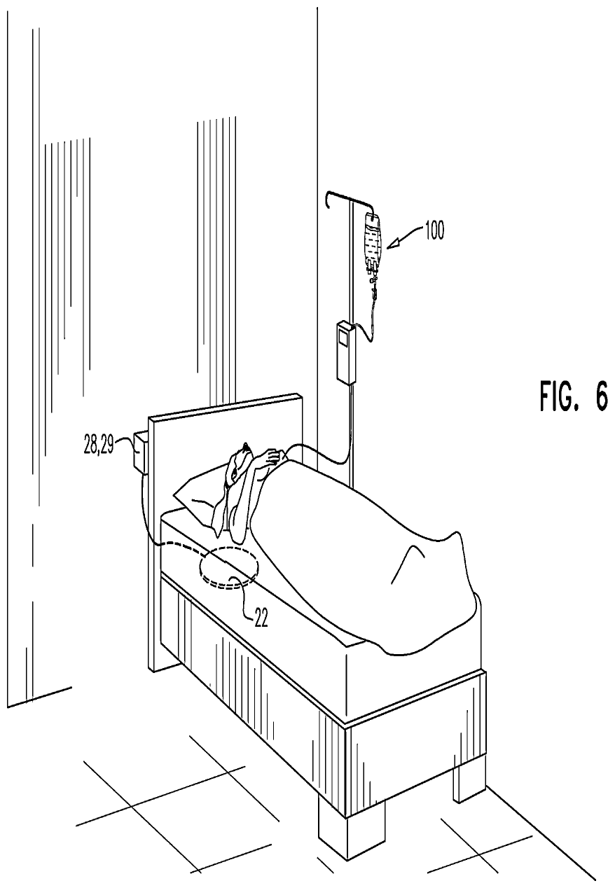

Reference is now made to FIG. 5, which is a flowchart showing steps that are performed by a computer processor in order to aid infirm subject 70, in accordance with some applications of the present invention. As described hereinabove, for some applications, a set of telecommunication devices that are associated with a given set of people other than the subject is designated. For example, such people may include friends, relatives, and/or caregivers of the infirm subject. For some applications, in step 90, a computer processor (such as the computer processor of telecommunications device 72 of subject 70, or a computer processor associated with a remote server) receives designations of respective telecommunication devices belonging to the set of telecommunication devices, as being associated with respective people. In step 92, the computer processor receives an indication that one of the set of telecommunication devices associated with the people other than the subject is disposed within a given distance of the subject's telecommunication device. For example, referring again to FIG. 3, the computer processor may be configured to detect that one of the telecommunications devices 76 is at a distance that is indicative of the other person 78 being at the door to the subject's room, or at the front door of the subject's home. For some applications, the telecommunications devices communicate with the subject's telecommunications devices via a communications protocol such as Bluetooth, Zigbee, etc. In step 94, at least partially in response to receiving the indication in step 92, the computer processor generates an output on the subject's telecommunication device, indicating the identity of the person associated with the telecommunication device that is within the given distance of the first telecommunication device.

For example, in response to someone ringing at the subject's front door, the subject may check an application on his/her phone to check whether the person at her door is one of the people who is designated as being one of his/her caregivers, e.g., a child, a grandchild, a nurse, etc. A computer processor associated with the application on his/her phone will check whether there is a telecommunications device within a given distance of the subject's phone that belongs to one of a designated group of caregivers. In response to detecting that there is such a device within the given distance, the computer processor drives the application to display a picture of the caregiver to whom the device belongs, and/or to display text on the subject's phone (or to generate an audio output) indicating the identity of the person to whom the telecommunications device belongs.

For some applications, if a service-providing organization is sending someone (e.g., a technician) to visit the infirm subject, the organization sends identification data to the computer processor (e.g., the computer processor of the subject's telecommunications device, or a remote computer processor). For example, Bluetooth identification data associated with the visitor's telecommunications device (e.g., phone) may be sent to the computer processor. Subsequently, when the visitor arrives at the subject's home, such that the visitor's telecommunications device is within a given distance of the subject's telecommunications device, the subject's telecommunications device communicates with the visitor's telecommunications device. Typically, the computer processor verifies that the telecommunications device that is within the given distance of the subject's telecommunications device is associated with the identification data. In response to verifying that the telecommunications device that is within the given distance of the subject's telecommunications device is associated with the identification data, the computer processor generates an output on the subject's telecommunications device indicating that it is safe to allow entry to the person at the door, and/or generates an output on the subject's telecommunications device indicating the identity of the person to whom the telecommunications device belongs.

Reference is now made to FIG. 6, which is a schematic illustration of sensor 22 disposed underneath a mattress of a subject, the subject being administered medication (which is typically pain-relief medication) by a medication-administration pump 100, in accordance with some applications of the present invention. Sensor 22 is typically as described hereinabove. Typically, the sensor is configured to generate a sensor signal and computer processor 28 is configured to analyze the sensor signal and to derive physiological parameters of the subject, based upon analyzing the sensor signal. For example, the computer processor may derive cardiac-related parameters (such as heart rate, heart-rate variability, heart rate patterns, etc.), and/or respiration-related parameters (such as respiration rate, respiration rate patterns, etc.).

For some applications, pump 100 is a patient-controlled analgesia pump. For some applications, in response to detecting that the subject's respiration rate passes a first threshold (e.g., in response to detecting that the subject's respiration rate is below a given threshold (such as, below between 8 breaths per minute and 5 breaths per minute), the computer processor prevents pump 100 from delivering the medication to the subject (e.g., because delivery of pain relief medication when the subject's respiration rate is below a given threshold may cause the subject to stop breathing). Typically, in response to detecting that the subject's respiration rate has not passed the threshold (e.g., in response to detecting that the subject's respiration rate is not below the given threshold) the computer processor allows pump 100 to deliver the medication to the subject.

Typically, the computer processor monitors physiological parameters of the subject over the course of one or more cycles of the subject (a) feeling pain, (b) receiving pain medication from pump 100, (c) the subject's pain level decreasing, and (d) the subject's pain level increasing as the effect of the medication diminishes. For example, the computer processor may monitor cardiac-related parameters (such as heart rate, heart-rate variability, heart rate patterns, etc.), and/or respiration-related parameters (such as respiration rate, respiration rate patterns, etc.). In response thereto, the computer processor determines a correspondence between changes in physiological parameters of the subject, and levels of pain that the subject is feeling. Subsequently, based upon monitoring the subject's physiological parameters, the computer processor automatically determines when the subject is undergoing pain that is at a level that is such that the subject requires pain-relief medication. In response thereto, the computer processor generates an alert (e.g., at a nurses' station, or on a telecommunications device of a caregiver). Alternatively or additionally, the computer processor automatically drives pump 100 to deliver pain relief medication, in response to determining that the subject is undergoing pain that is at a level that is such that the subject requires pain-relief medication. For some applications, the computer processor prevents pump 100 from delivering medication, even in response to the subject requesting pain-relief medication, for example, in response to detecting (based on the detected physiological parameters) that the subject is not undergoing pain, or in not undergoing pain that is sufficient to merit administering the medication.

For some applications, pump 100 is an infusion pump. Typically, the computer processor monitors physiological parameters of the subject while medication is administered to the subject via the infusion pump. For example, the computer processor may monitor cardiac-related parameters (such as heart rate, heart-rate variability, heart rate patterns, etc.), and/or respiration-related parameters (such as respiration rate, respiration rate patterns, etc.). For some applications, the computer processor derives a response of one or more physiological parameters of the subject to the administration of the medication, and compares the subject's response to a historical response of the subject to the medication, and/or to responses of other patients to the administration of the medication. For example, the computer processor may compare the subject's response to the responses of other patients, e.g., other patients in a similar demographic group to the subject (such as, patients in the same age group as the subject, of the same gender as the subject, etc.). A difference between the subject's response to the administration of the medication, and a historical response of the subject to the medication, and/or responses of other patients to the administration of the medication, may indicate that the subject is having an adverse reaction to the medication, that the wrong medication is being administered, that the wrong dosage of medication is being administered, and/or another clinical issue with the administration of the medication to the subject.

For some applications, in response to identifying that a response of the physiological parameters of the subject to the administration of the medication is different from a historical response of the subject to the medication, and/or is different from responses of other patients, the computer processor generates an alert (e.g., at a nurses' station, or on a telecommunications device of a caregiver), and/or prevents the pump from administering the medication to the subject. Typically, the computer processor generates the alert in response to the response of the physiological parameters of the subject being different from the historical response of the subject, and/or from responses of other patients more than a threshold amount.

In general, computer processor 28 may be embodied as a single computer processor 28, or a cooperatively networked or clustered set of computer processors. Computer processor 28 is typically a programmed digital computing device comprising a central processing unit (CPU), random access memory (RAM), non-volatile secondary storage, such as a hard drive or CD ROM drive, network interfaces, and/or peripheral devices. Program code, including software programs, and data are loaded into the RAM for execution and processing by the CPU and results are generated for display, output, transmittal, or storage, as is known in the art. Typically, computer processor 28 is connected to one or more sensors via one or more wired or wireless connections. Computer processor 28 is typically configured to receive signals (e.g., motion signals) from the one or more sensors, and to process these signals as described herein.

In the context of the claims and specification of the present application, the term “motion signal” is used to denote any signal that is generated by a sensor, upon the sensor sensing motion. Such motion may include, for example, respiratory motion, cardiac motion, or other body motion, e.g., large body-movement. Similarly, the term “motion sensor” is used to denote any sensor that senses motion, including the types of motion delineated above.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purposes of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 28) coupled directly or indirectly to memory elements (e.g., memory 29) through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that each block of the flowcharts shown in FIGS. 2, 4, and 5, and combinations of blocks in the flowcharts, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the flowcharts and/or algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowcharts and/or algorithms described in the present application.

Computer processor 28 is typically a hardware device programmed with computer program instructions to produce a special purpose computer. For example, when programmed to perform the algorithms described with reference to FIG. 2, computer processor 28 typically acts as a special purpose ovulation-prediction computer processor, and when programmed to perform the algorithms described with reference to FIGS. 4 and 5, computer processor 28 typically acts as a special purpose infirm-subject-monitoring computer processor. Typically, the operations described herein that are performed by computer processor 28 transform the physical state of memory 29, which is a real physical article, to have a different magnetic polarity, electrical charge, or the like depending on the technology of the memory that is used.

Techniques described herein may be practiced in combination with techniques described in one or more of the following patents and patent applications, which are incorporated herein by reference. In some applications, techniques and apparatus described in one or more of the following patents and patent applications, which are incorporated herein by reference, are combined with techniques and apparatus described herein:
U.S. patent application Ser. No. 11/048,100, filed Jan. 31, 2005, which issued as U.S. Pat. No. 7,077,810;
U.S. patent application Ser. No. 11/197,786, filed Aug. 3, 2005, which issued as U.S. Pat. No. 7,314,451;
U.S. patent application Ser. No. 11/446,281, filed Jun. 2, 2006, which issued as U.S. Pat. No. 8,376,954;
U.S. patent application Ser. No. 11/552,872, filed Oct. 25, 2006, now abandoned, which published as US 2007/0118054;
U.S. patent application Ser. No. 11/755,066, filed May 30, 2007, now abandoned, which published as US 2008/0114260;
U.S. patent application Ser. No. 11/782,750, filed Jul. 25, 2007, which issued as U.S. Pat. No. 8,403,865;
U.S. patent application Ser. No. 12/113,680, filed May 1, 2008, now abandoned, which published as US 2008/0275349;
U.S. patent application Ser. No. 12/842,634, filed Jul. 23, 2010, which issued as U.S. Pat. No. 8,517,953;
U.S. patent application Ser. No. 12/938,421, filed Nov. 3, 2010, which issued as U.S. Pat. No. 8,585,607;
U.S. patent application Ser. No. 12/991,749, filed Nov. 9, 2010, which issued as U.S. Pat. No. 8,821,418;
U.S. patent application Ser. No. 13/107,772, filed May 13, 2011, which issued as U.S. Pat. No. 8,491,492;
U.S. patent application Ser. No. 13/305,618, filed Nov. 28, 2011, now abandoned, which published as US 2012/0132211;
U.S. patent application Ser. No. 13/389,200, filed Jun. 13, 2012, now abandoned, which published as US 2012/0253142;
U.S. patent application Ser. No. 13/750,957, filed Jan. 25, 2013, which issued as U.S. Pat. No. 8,603,010;
U.S. patent application Ser. No. 13/750,962, filed Jan. 25, 2013, which issued as U.S. Pat. No. 8,679,034;
U.S. patent application Ser. No. 13/863,293, filed Mar. 15, 2013, now abandoned, which published as US 2013/0245502;
U.S. patent application Ser. No. 13/906,325, filed May 30, 2013, which issued as U.S. Pat. No. 8,882,684;
U.S. patent application Ser. No. 13/921,915, filed Jun. 19, 2013, which issued as U.S. Pat. No. 8,679,030;
U.S. patent application Ser. No. 14/019,371, filed Sep. 5, 2013, which published as US 2014/0005502;
U.S. patent application Ser. No. 14/020,574, filed Sep. 6, 2013, which issued as U.S. Pat. No. 8,731,646;
U.S. patent application Ser. No. 14/054,280, filed Oct. 15, 2013, which issued as U.S. Pat. No. 8,734,360;
U.S. patent application Ser. No. 14/150,115, filed Jan. 8, 2014, which issued as U.S. Pat. No. 8,840,564;
U.S. patent application Ser. No. 14/231,855, filed Apr. 1, 2014, which issued as U.S. Pat. No. 8,992,434;
U.S. patent application Ser. No. 14/454,300, filed Aug. 7, 2014, which issued as U.S. Pat. No. 8,942,779;
U.S. patent application Ser. No. 14/458,399, filed Aug. 13, 2014, which issued as U.S. Pat. No. 8,998,830;
U.S. patent application Ser. No. 14/474,357, filed Sep. 2, 2014, which published as US 2014/0371635;
U.S. patent application Ser. No. 14/557,654, filed Dec. 2, 2014, issued as U.S. Pat. No. 9,026,199;
U.S. patent application Ser. No. 14/631,978, filed Feb. 26, 2015, published as US 2015/0164438;
U.S. patent application Ser. No. 14/624,904, filed Feb. 18, 2015, published as US 2015/0164433;
U.S. patent application Ser. No. 14/663,835, filed Mar. 20, 2015, published as US 2015/0190087;
U.S. patent application Ser. No. 14/810,814, filed Jul. 28, 2015, published as US 2015/0327792;
U.S. patent application Ser. No. 15/431,842, filed Feb. 14, 2017, published as US 2017/0231545;
International Patent Application PCT/IL2005/000113, which published as WO 2005/074361;
International Patent Application PCT/IL2006/000727, which published as WO 2006/137067;
International Patent Application PCT/IL2006/002998, which published as WO 2007/052108;
International Patent Application PCT/IL2008/000601, which published as WO 2008/135985;
International Patent Application PCT/IL2009/000473, which published as WO 2009/138976;
International Patent Application PCT/IL2011/050045, which published as WO 2012/077113;
International Patent Application PCT/IL2013/050283, which published as WO 2013/150523;
International Patent Application PCT/IL2014/050644, which published as WO 2015/008285;
International Patent Application No. PCT/IL2015/050880 to Shinar, which published as WO 16/035073; and
International Patent Application No. PCT/IL2017/050182 to Katz, which published as WO 17/138005.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for use with a first telecommunication device that is associated with a subject requiring care, and a set of telecommunication devices that are associated with people other than the subject, the apparatus comprising:
at least one computer processor configured:
to detect whether any of the set of telecommunication devices associated with the people other than the subject are disposed within a given distance of the first telecommunication device; and
in response to detecting that, over a given time period, none of the set of telecommunication devices associated with people other than the subject is disposed within a given distance of the first telecommunication device, to generate an alert.

2. The apparatus of claim 1, wherein the set of telecommunication devices associated with the people other than the subject includes telecommunication devices associated with any person other than the subject, and wherein the at least one computer processor is configured to generate the alert in response to detecting that, over the given time period, no telecommunication device associated with any person other than the subject is disposed within the given distance of the first telecommunication device.

3. The apparatus of claim 1, wherein the set of telecommunication devices associated with the people other than the subject includes telecommunication devices associated with a set of caregivers that are assigned to the subject, and wherein the at least one computer processor is configured to generate the alert in response to detecting that, over the given time period, none of the set of telecommunication devices associated with the set of caregivers that are assigned to the subject is disposed within the given distance of the first telecommunication device.

4. An apparatus for use with a first telecommunication device that is associated with a subject requiring care, and a second of telecommunication devices associated with people other than the subject, the apparatus comprising:
at least one computer processor configured:
to receive designations of respective telecommunication devices belonging to the set of telecommunication devices, as being associated with respective people;
to receive an indication that one of the telecommunication devices belonging to the set of telecommunication devices is within a given distance of the first telecommunication device; and
at least partially in response thereto, to generate an output on the first telecommunication device, indicating an identity of the person associated with the telecommunication device that is within a given distance of the first telecommunication device.

5. The apparatus of claim 1, wherein the at least one computer processor is included in the first telecommunication device of the subject.

6. The apparatus of claim 1, wherein the at least one computer processor is included in a server that is remote from the subject.

7. The apparatus of claim 1, wherein the at least one computer processor is included in one of the set of telecommunication devices that are associated with people other than the subject.

8. The apparatus of claim 1, wherein the first telecommunication device and each telecommunication device of the set of telecommunications devices that are associated with people other than the subject each comprises a phone.

9. The apparatus of claim 1, wherein the first telecommunication device and each telecommunication device of the set of telecommunications devices that are associated with people other than the subject each comprises one of the following: a computer, a laptop computer, or a tablet device.

10. The apparatus of claim 1, wherein to detect whether any of the set of telecommunication devices associated with the people other than the subject are disposed within a given distance of the first telecommunication device involves the use of communications between the first telecommunication device and one or more of the set of telecommunication devices according to a Bluetooth protocol or a Zigbee protocol.

11. The apparatus of claim 1, wherein the given time period comprises once a day, once a week, or once every few hours.

12. The apparatus of claim 1, wherein the alert is generated on a telecommunications device of the subject's primary caregiver.

13. The apparatus of claim 1, wherein the alert is generated at a monitoring center.

14. The apparatus of claim 1, wherein the computer processor is configured to detect whether a telecommunications device other than the set of telecommunication devices is within the given distance of the first telecommunications device, and to generate a presence alert in response thereto.

15. The apparatus of claim 1, wherein the computer processor is configured to generate an output on the first telecommunication device to indicate an identity of a person associated with any of the set of telecommunication devices that are within the given distance of the first telecommunication device.

16. The apparatus of claim 4, wherein the output comprises a picture of the person associated with the telecommunication device that is within the given distance of the first telecommunication device.

17. The apparatus of claim 4, wherein the output comprises an audio output identifying the person associated with the telecommunication device that is within the given distance of the first telecommunication device.

18. The apparatus of claim 4, wherein the output comprises text on a display of the first telecommunication device identifying the person associated with the telecommunication device that is within the given distance of the first telecommunication device.

19. The apparatus of claim 4, wherein the at least one computer processor is communicatively coupled to an infusion pump that is operable to deliver medication to the subject.

20. The apparatus of claim 18, wherein the at least one computer processor is also communicatively coupled to a physiological sensor that detects the subject's heart rate and/or respiration rate.
